(19) United States
(12) Patent Application Publication
(10) Pub. No.: US 2022/0280350 A1
(43) Pub. Date: Sep. 8, 2022

(54) ABSORBENT ARTICLE

(71) Applicant: The Procter & Gamble Company, Cincinnati, OH (US)

(72) Inventors: Emma Lynn Sartini, Covington, KY (US); Alexzandra Joan Ramachandran, Cincinnati, OH (US)

(21) Appl. No.: 17/688,959

(22) Filed: Mar. 8, 2022

Related U.S. Application Data
(60) Provisional application No. 63/158,054, filed on Mar. 8, 2021.

(51) Int. Cl. A61F 13/42; A61F 13/47; A61F 13/53; A61F 13/513; A61F 13/514

(52) U.S. Cl. CPC A61F 13/42; A61F 13/47; A61F 13/53; A61F 13/51394; A61F 13/51496; A61F 2013/422

(57) ABSTRACT

A disposable absorbent article is described. The disposable absorbent article has a topsheet; a backsheet; and an absorbent system disposed therebetween. The absorbent system has an absorbent core having longitudinal side edges on opposite sides of the absorbent core and end edges on opposite sides of the absorbent core. The absorbent core is disposed in an absorbent core zone. The absorbent article also has an outer periphery formed at least in part by a portion of the topsheet and a portion of the backsheet; an outer boundary zone including the outer periphery. The absorbent article also has an inner boundary zone disposed between the outer boundary zone and the absorbent core zone in a rear portion of the absorbent article. There is a visible difference between the absorbent core zone and the inner boundary zone as well as the absorbent core zone and the outer boundary zone.

### CROSS REFERENCE TO RELATED APPLICATION

This application claims the benefit of U.S. Provisional Application No. 63/158,054, filed Mar. 8, 2021, the substance of which is incorporated herein by reference.

### FIELD OF THE INVENTION

The present invention pertains to disposable absorbent articles having a color profile to create visual cues to the user.

### BACKGROUND OF THE INVENTION

Disposable absorbent articles such as feminine hygiene pads have been utilized for quite some time. These absorbent articles are utilized to absorb liquid insults, i.e. body exudates. In general, such absorbent articles comprise a topsheet, a backsheet, and an absorbent core disposed between the topsheet and the backsheet.

There are a wide variety of absorbent articles designed to absorb body exudates. These absorbent articles, even when focusing on menstrual pads or adult incontinence pads, come in many shapes and sizes. Their absorbent capacities vary to quite a large extent. So the amount of time that a user can utilize an absorbent article very much depends on the absorbent capacity of the article.

An additional complicating factor is the amount of liquid discharged to the absorbent article. As an example, during menstruation, the amount of discharge is not constant on a day to day basis. So, the amount of time an absorbent article lasted on day one may be quite different from the amount of time the absorbent article lasts on day 4 of the menstruation cycle. The same is true for adult incontinence pads. The amount of time before these pads reach capacity can vary greatly depending on the amount of liquid insult to the pad.

While replacing feminine hygiene pads, including menstrual pads and adult incontinence pads, in a timely manner can help preclude leakage. Placement of the pad within the underwear is just as important. Misaligned pads can cause leakage problems even prior to the pad reaching its full absorbent capacity. This can not only frustrate the user but may also cause quite a bit of embarrassment.

Based on the foregoing, what is needed is an absorbent article which provides the user with visual cues regarding both capacity and placement. Additionally, it would be beneficial to provide such cues in a simplified manner such that the complexity of manufacturing such articles is not overly complex.

### SUMMARY OF THE INVENTION

Disposable absorbent articles constructed in accordance with the present disclosure comprise a longitudinal axis and a transverse axis disposed perpendicularly to the longitudinal axis, a front portion, a rear portion, and a central portion disposed between the front portion and rear portion. In one particular example, the disposable absorbent article may further comprise a topsheet; a backsheet; an absorbent system disposed between the topsheet and the backsheet, wherein the absorbent system comprises an absorbent core having longitudinal side edges on opposite sides of the absorbent core and end edges on opposite sides of the absorbent core, the absorbent core being disposed in an absorbent core zone, wherein the absorbent core zone exhibits a first L* value; an outer periphery formed at least in part by a portion of the topsheet and a portion of the backsheet; an outer boundary zone comprising the outer periphery, the outer boundary zone having a width of less than 30 mm, wherein the outer boundary zone exhibits a second L* value of less than 89; an inner boundary zone disposed between the outer boundary zone and the absorbent core zone in the rear portion, the inner boundary zone exhibits a third L* value which is greater than the second L* value; and wherein the first L* value is greater than the second and third L* values such that there is a perceptible visual difference between the absorbent core zone and the inner boundary zone.

In another example, the disposable absorbent articles may further comprise: a topsheet; a backsheet; an absorbent system disposed between the topsheet and the backsheet, wherein the absorbent system comprises an absorbent core having longitudinal side edges on opposite sides of the absorbent core and end edges on opposite sides of the absorbent core, the absorbent core being disposed in an absorbent core zone; an outer periphery formed at least in part by a portion of the topsheet and a portion of the backsheet; an outer boundary zone comprising the outer periphery, the outer boundary zone having a width of less than 30 mm; an inner boundary zone disposed between the outer boundary zone and the absorbent core zone in the rear portion; and wherein a first delta E* between the absorbent core zone and the inner boundary zone is greater than greater than 4, more preferably greater than 7, or most preferably greater than 9.

### BRIEF DESCRIPTION OF THE DRAWINGS

FIG. 1A is a picture of an absorbent article showing a wearer-facing surface of the absorbent article, wherein the absorbent article is constructed in accordance with the present disclosure.

FIG. 1B is cross section of the absorbent article of FIG. 1A taken along line 1B-1B.

FIG. 1C is a cross section of the absorbent article of FIG. 1A taken along line 1C-1C.

FIG. 2 is a picture of the absorbent article of FIG. 1A showing a garment-facing surface of the absorbent article.

FIG. 3 is a schematic representation of a print portion shown on the garment-facing surface of the absorbent article of FIG. 1A.

### DETAILED DESCRIPTION OF THE INVENTION

The term “absorbent article” as used herein refers to devices which absorb and contain exudates, and, more specifically, refers to devices which are placed against or in proximity to the body of the wearer to absorb and contain the various exudates discharged from the body. Absorbent articles of the present disclosure include, but are not limited to, diapers, adult incontinence briefs, training pants, diaper holders, menstrual pads, incontinence pads, liners, absorbent inserts, pantiliners, tampons, and the like.

Absorbent articles of the present disclosure provide the user with placement guides along with highlighted edges of an absorbent core within the absorbent article. The placement guide provides input to the user regarding positioning of the absorbent article on their underwear. Correct placement of the article on the underwear reduces the likelihood of leakage. And, the highlighted edges of the absorbent core can serve as a capacity cue to the user. The capacity cue can provide a signal to the user when the pad is running out of capacity and should be changed. Similar to the placement guides, this feature also reduces the likelihood of leakage.

Absorbent articles of the present disclosure comprise a topsheet, a backsheet, and an absorbent system disposed between the topsheet and the backsheet. The absorbent articles of the present disclosure further comprise a longitudinal axis and a transverse axis extending generally perpendicular to the longitudinal axis. Additionally, absorbent articles of the present disclosure comprise a front portion, a rear portion, and a central portion disposed between the front portion and the rear portion. Each of the front portion, rear portion, and central portion, comprise about one third of an overall length of the absorbent article.

The absorbent article comprises an outer periphery or outer edge. The outer periphery defines the extent of the absorbent article both longitudinally and transversely. The outer periphery typically comprises the outer edge of the absorbent article where the topsheet is joined to the backsheet. The topsheet may be joined to backsheet in any suitable manner, including adhesives, heat sealing, the like, or combinations thereof.

As mentioned previously, the absorbent system is disposed between the topsheet and the backsheet. In addition to comprising the absorbent core, the absorbent system may further comprise additional optional layers. For example, an acquisition/distribution layer may be provided between the topsheet and the backsheet. As another example, a layer which quickly acquires liquid insults from the topsheet may be provided between the topsheet and the absorbent core while a separate primary distribution layer may be provided between the acquisition layer and the absorbent core. As yet another example, a secondary distribution layer may be provided between the absorbent core and the backsheet. This secondary distribution layer may be provided in addition to the acquisition/distribution layer or in addition to the primary acquisition layer and the distribution layer.

In order to provide the capacity and placement cues of the absorbent articles of the present disclosure, one or more colorants may be utilized. For example, the capacity cue may comprise a first colorant which is visible from a wearer-facing side of the absorbent article. As another example, the placement cue may comprise a second colorant which is visible from a garment-facing side of the article. The first colorant may be provided on layers of the absorbent article which are more proximal to the topsheet. As an example, in order to highlight the longitudinal side edges and/or the transverse end edges of the absorbent core, the backsheet, one or more layers between the absorbent core and the backsheet, and/or one or more layers between the topsheet and the absorbent core, may comprise a colorant.

The second colorant may be provided on layers of the absorbent article which are more proximal to the backsheet. As an example, the second colorant may be provided on a wearer-facing and/or a garment-facing surface of the backsheet and/or a secondary distribution layer.

While the first colorant and second colorant may be provided as described previously, such applications of colorant can create manufacturing complexities in that there are two separate layers which comprise printing which can mean increased costs due to the amount of colorant being provided. However, forms are contemplated where colorant associated with one or more layers of the absorbent articles may be utilized to accommodate both the capacity cue as well as the placement cue.

In order to further understand this simplified form, some information regarding zones of the absorbent article must be discussed. Absorbent articles of the present disclosure comprise a plurality of zones. One of the primary zones is the absorbent core zone. The absorbent core zone is defined by longitudinal side edges and end edges of the absorbent core. The absorbent core zone includes portions of all layers of the absorbent article which overlap the absorbent core and are within the bounds of the longitudinal side edges and transverse end edges of the absorbent core.

The absorbent articles of the present disclosure similarly comprise an outer boundary zone and an inner boundary zone. The outer boundary zone comprises the outer periphery of the absorbent article and extends inboard thereof. For example, the outer boundary zone can extend to about 30 mm or less inboard of the outer periphery. In one specific example, the outer boundary zone may comprise the periphery of the absorbent article in the rear portion of the absorbent article. Additionally, the outer boundary zone may comprise the periphery, or a portion of the periphery in the central region of the absorbent article.

The outer boundary zone may comprise a plurality of layers of the absorbent article. As an example, the outer boundary zone may comprise the topsheet and the backsheet. The outer boundary zone may comprise the acquisition/distribution layer, primary distribution layer, and/or secondary distribution layer.

The inner boundary zone is disposed between the outer boundary zone and the absorbent core zone. The inner boundary zone comprises a portion of all of the layers of the absorbent article within the inner boundary zone, with the exception of the absorbent core. Much like the outer boundary zone, the inner boundary zone may comprise the topsheet and the backsheet and additionally may comprise the acquisition/distribution layer, primary distribution layer, and/or secondary distribution layer. The inner boundary zone may be provided mainly in the rear portion of the absorbent article. For example, the inner boundary zone may have a larger extent, e.g. larger surface area, in the rear portion of the absorbent article than in the central portion and/or front portion of the absorbent article.

In addition to the layers of the absorbent article mentioned heretofore or independently thereof, another layer may be provided. For example, a support layer may be disposed between the absorbent core and the backsheet or between the topsheet and the backsheet. The support layer may be any suitable material, e.g. nonwoven, film, or combinations thereof. Support layers may be particularly useful in pads which are designed for overnight use.

The support layer may be disposed, in part, in the absorbent core zone, inner boundary zone, and optionally the outer boundary zone. Additionally, the support layer may be disposed in the rear portion and potentially partially in the central portion. In some forms, the support layer may be absent from the front portion. Support layers as well as overnight pads are described in additional detail in U.S. Pat. No. 8,715,258 B2, issued to Munakata et al.

With the understanding of the zones of the absorbent article, we now return to the use of colorant to provide the capacity and placement cues. As noted, a colorant associated with one or more layers can provide both the capacity and placement cues. However placement of the colorant is critical to accommodating both cues. For example, colorant which is disposed in an absorbent core zone, e.g. between the absorbent core and the backsheet, i.e. viewable from the garment-facing surface, may be sufficient as a placement cue but may not sufficiently highlight the edges of the absorbent core to provide a capacity cue. Similarly, colorant provided in the absorbent core zone viewable from the wearer-facing surface of the article may sufficiently highlight the edges of the core but may not be visible from the garment-facing surface of the absorbent article.

In general, the absorbent core is opaque and does not allow for colorants disposed on one side of the absorbent core to be viewed on the opposite side. So, for the absorbent article of the present disclosure, colorant can be provided in the inner boundary zone and the outer boundary zone. Colorant provided in the inner boundary zone can provide both the capacity cue and the placement cue. It is also worth noting that more than one colorant may be utilized for the placement and/or capacity cue.

In order for the colorant to be viewed from the wearer-facing surface or the garment-facing surface, the materials within the inner boundary zone and/or outer boundary zone should be selected such that they are not overly opaque and inhibit viewing of the colorant. As an example, several topsheet materials were tested to understand the impact of opacity on the perception of the cues—specifically the capacity cue. Data for the samples tested is provided in Table 1 below.

TABLE 1

| | Basis Weight | | Perception of |
|---|---|---|---|
| Sample Description | (grams/meter\hat{ }2) | Opacity | Visual Queue |
| Sample 1—NW | 24 | 42.6 | pass |
| (non-apertured region) | | | |
| Sample 1—NW | 24 | 41.4 | pass |
| (apertured region) | | | |
| Sample 2—NW | 25 | 27.6 | Pass |
| Sample 3—NW | 40 | 56.9 | Fail |
| Sample 4—NW | 40 | 57.1 | Fail |
| Sample 5—NW | 35 | 51.4 | pass |
| Sample 6—NW | 24 | 36.8 | Pass |
| Sample 7—NW | 24 | 41.6 | Pass |
| Sample 8—NW | 24 | 42.2 | pass |
| Sample 9—NW | 42 | 58.8 | Fail |

Sample 1 was a carded nonwoven material comprising 100 percent bico fibers having a denier of 2, which were polyethylene (PE)/polyethylene terephthalate (PET) with 1.5 percent titanium dioxide.

Sample 2 was a nonwoven material comprising 100 percent bico fibers which were polypropylene (PP) and PE with 1.5 percent titanium dioxide.

Sample 3 was a nonwoven material comprising 1.5 denier bico fibers of PE and PET with 1.5 percent titanium dioxide.

Sample 4 was a nonwoven having 2 denier bico fibers of PE and PET with 1.5 percent titanium dioxide.

Samples 5 and 6 were nonwovens having 3 denier spiral bico fibers of PE/PET and 2 denier bico fibers of PE/PET, with 1.5 percent titanium dioxide.

Sample 7 was a nonwoven material having 2.5 denier bico fibers of PE/PET and 2 denier bico fibers of PE/PET, with 1.5 percent titanium dioxide.

Sample 8 was a nonwoven material having 2.5 denier spiral bico fibers of PE/PET and 2 denier bico fibers of PE/PET, with 1.5 percent titanium dioxide.

Sample 9 was a nonwoven material comprising 100 percent bico fibers having a denier of 2, which were polyethylene (PE)/polyethylene terephthalate (PET) with 1.5 percent titanium dioxide. While Sample 9 had a similar material construction to that of Sample 1, Sample 9 comprises an aperture pattern which was a floral pattern.

It is worth noting that film topsheets may also be utilized as long as they have a similar opacity as what is demonstrated in Table 1 regarding those materials which achieved a score of “pass.” The score of “pass” is a subjective metric which focuses on a materials ability to allow underlying colorant to be viewed by the user. So for those materials which received a score of “fail,” it was determined that their opacity was too high to allow for the edges of the absorbent core to be highlighted sufficiently.

While the basis weights shown in Table 1 are nominal (target basis weights), it is believed that the actual basis weights are within plus or minus 5 percent of the target basis weight. Based on the data in Table 1, the inventors believe that the topsheet should have a basis weight of no greater than 35 gsm in order to meet allow sufficient visibility for the capacity cue. The topsheet may have a basis weight of 35 gsm or less, more preferably 30 gsm or less, or most preferably 27 gsm or less, specifically reciting all values within these ranges and any ranges created thereby.

Topsheets of the present disclosure can have an opacity of less than 53, more preferably less than 51, or most preferably less than 47, specifically reciting all values within these ranges and any ranges created thereby. And while in theory opacities which are very low would tend to facilitate the visualization of the cues mentioned heretofore, topsheets with very low opacities will also tend to show more vividly the staining caused by the liquid insults. This can be very disturbing to some users, particularly where the liquid insult is menses. So, the topsheets of the present disclosure should have a sufficient level of opacity to allow for the visualization of the cues but also to sufficiently mask staining caused by the liquid insults to the absorbent article. With this in mind, topsheets of the present disclosure can have an opacity of from between about 20 to about 53, more preferably from about 23 to about 51, or most preferably from about 25 to about 47, specifically reciting all values within these ranges and any ranges created thereby.

It is further believed that the foregoing opacity values may similarly apply to multiple layers of the absorbent article. For example, where a support layer is disposed between the topsheet and the absorbent core and the colorant is disposed on the backsheet, collectively, the support layer and the topsheet can be within the opacity ranges described heretofore. In this same example, where the colorant is disposed on a wearer-facing surface of the backsheet, the backsheet may have similar opacity values discussed herein such that the placement cue is easily visible to the user. In yet another example where the colorant is disposed on the garment-facing surface of the absorbent article, the collective opacity of the backsheet, optional layers between the topsheet and the backsheet, and the topsheet can have the opacity values described herein. In such constructions, as noted previously, it may be beneficial to select the topsheet such that its opacity is higher than that of the optional layers as well as that of the backsheet. Or alternatively, the topsheet and backsheet may be selected such that their opacities are similar while the optional layers between the topsheet and the backsheet have lower opacity than that of either the topsheet or the backsheet. Selection of higher opacity topsheets and/or backsheets can help mask stains within the pad.

With the topsheet and any other intervening layers chosen as described above, the absorbent core zone, inner boundary zone, and outer boundary zone may have distinct visual appearances. These distinct visual appearances set up the cues discussed herein. The differences in visual appearance between the absorbent core zone, inner boundary zone, and outer boundary zone, are described herein within the context of the Hunter color scale. In the Hunter color scale, several values are measured. The lightness or darkness of a sample is measured as L*. Higher values of L* are indicative of lighter colors while lower values are indicative of darker colors. Red versus green of a sample is measured as a*. A positive number indicates red and a negative number indicates green. Yellow versus blue of a sample is measured as b*. A positive number indicates yellow and a negative number indicates blue. The distance between two colors in color space is denoted as delta E* or ΔE*. Within the context of disposable absorbent articles, it is believed that visible differences between samples occurs at as low as a ΔE* of 2. Delta E* is discussed in additional detail in the TEST METHODS section of this specification.

The absorbent core zone will generally comprise a background color, e.g. white. So, the inner boundary zone should comprise a contrasting color, e.g. a difference in a*, b*, L* or delta E*. Similarly, in order to simplify the creation of the cues, the outer boundary zone can have a contrasting color value to that of the inner boundary zone and the absorbent core zone. For example, the absorbent core may exhibit a first L* value, the outer boundary zone may exhibit a second L* value, and the inner boundary zone may exhibit a third L* value. The second L* value can be less than about 89. And, the third L* value can be greater than the second L* value, and the first L* value can be greater than the second L* value.

As noted, the absorbent core zone typically comprises a background color, e.g. white. So, the first L* value can typically be fairly high. As an example, the first L* value can be greater than about 94 or between 94 to 97. Note while the first L* value may be lower than 94, in such constructions, the third L* value should be lower than the first L* value by at least 4 to ensure that the inner boundary zone is visibly distinct from the absorbent core zone. The difference in L* values between the absorbent core zone and the inner boundary zone can ensure that the edges of the absorbent core are highlighted sufficiently to provide the visual capacity cue as described herein.

The inner boundary zone comprises the third L* value which is lower than the first L* value and greater than the second L* value. The third L* value can be between 60 and 94, more preferably from between about 65 to about 92, or most preferably from about 70 to about 90.

The outer boundary zone comprises the second L* value which is lower than both the first L* value and the second L* value. The second L* value can be between about 50 to about 91, more preferably from about 50 to about 89, or most preferably from about 50 to about 83.

As noted previously, coupled with the difference in L* values, the absorbent core zone, inner boundary zone, and outer boundary zone, may comprise differences in the green—red measurement and yellow—blue measurement as well. As an example, a first delta E* value between the absorbent core zone and the inner boundary zone can be 4 or more, more preferably 7 or more, or most preferably 9 or more, specifically reciting all values within this range and any range created thereby. For example, the first delta E* can be between about 4 to about 18, or more preferably from about 4 to about 15, or most preferably from about 4 to about 13, specifically reciting all values within these ranges and any ranges created thereby.

Similarly a second delta E* between the inner boundary zone and the outer boundary zone can be greater than 4, more preferably greater than 6, or most preferably greater than 8. The first delta E* may be less than the second delta E*. In contrast, in some forms, the first delta E* may be greater than the second delta E*. Exemplary absorbent articles constructed in accordance with the present disclosure were created and compared to an absorbent article not in accordance with the present disclosure. The results are provided in Table 2 below.

TABLE 2

| Sample No. | L* | a* | b* | C* |
|---|---|---|---|---|
| Sample 1—wings | 72.37 | 5.81 | −16.47 | 17.5 |
| Sample 1—inner boundary zone | 87.82 | 1.03 | −4.12 | 4.2 |
| Sample 1—absorbent core zone | 96.05 | −0.63 | 3.05 | 3.1 |
| Sample 1—outer boundary zone | 80.23 | 3.24 | −9.65 | 10.2 |
| Sample 2—wings | 87.05 | 1.90 | −7.49 | 7.7 |
| Sample 2—inner boundary zone | 92.58 | −0.01 | −1.87 | 1.9 |
| Sample 2—absorbent core zone | 95.97 | −0.57 | 3.29 | 3.3 |
| Sample 2—outer boundary zone | 88.78 | 0.84 | −4.39 | 4.5 |
| Sample 3—wings | 90.5 | 0.5 | −4.01 | 4.0 |
| Sample 3—inner boundary zone | 94.5 | −0.56 | −0.32 | 0.6 |
| Sample 3—absorbent core zone | 94.85 | −0.58 | 2.81 | 2.9 |
| Sample 3—outer boundary zone | 89.77 | 0.53 | −3.45 | 3.5 |

Table 3 below shows the differences in L*, a*, b*, C*, as well as the ΔE* between various locations on the sample articles.

TABLE 3

| Sample No. and location | ΔL* | Δa* | Δb* | ΔC* | ΔE* |
|---|---|---|---|---|---|
| Sample 1—wings/inner | −15.45 | 4.78 | −12.35 | −13.22 | 20.35 |
| boundary zone | | | | | |
| Sample 1—absorbent core | 8.23 | −1.66 | 7.17 | −1.13 | 11.04 |
| zone/inner boundary zone | | | | | |
| Sample 1—inner boundary | 7.59 | −2.21 | 5.53 | −5.94 | 9.65 |
| zone/outer boundary zone | | | | | |
| Sample 1—absorbent core | 15.82 | −3.97 | 12.70 | −7.07 | 20.65 |
| zone/outer boundary zone | | | | | |
| Sample 2—wings/inner | −5.53 | 1.91 | −5.62 | −5.86 | 8.11 |
| boundary zone | | | | | |
| Sample 2—absorbent core | 3.39 | −0.51 | 5.16 | 1.46 | 6.19 |
| zone/inner boundary zone | | | | | |
| Sample 2—inner boundary | 3.80 | −0.85 | 2.52 | −2.60 | 4.64 |
| zone/outer boundary zone | | | | | |
| Sample 2—absorbent core | 7.19 | −0.32 | 7.68 | −1.14 | 10.52 |
| zone/outer boundary zone | | | | | |
| Sample 3—wings/inner | −3.55 | 1.06 | −3.69 | −3.40 | 5.23 |
| boundary zone | | | | | |
| Sample 3—absorbent core | 0.8 | −0.02 | 3.13 | 2.22 | 3.23 |
| zone/inner boundary zone | | | | | |
| Sample 3—inner boundary | 4.28 | −1.09 | 3.13 | −2.85 | 5.42 |
| zone/outer boundary zone | | | | | |
| Sample 3—absorbent core | 5.08 | −1.11 | 6.26 | −0.63 | 8.14 |
| zone/outer boundary zone | | | | | |

Samples 1 and 2 were constructed in accordance with the present disclosure and Sample 3 was not. Each of Samples 1-3 comprised a 24 gsm topsheet and a film backsheet. Between the film backsheet was a 50 gsm spunlace secondary topsheet and an airlaid absorbent core having a basis weight of 69 gsm. The backsheets for Samples 1 and 2 comprised a higher basis weight of colorant than did the backsheet for Sample 3.

The colorant utilized to create the cues described herein may be provided on any suitable layer or combination of layers of the absorbent article. Additionally, colorant(s) may be utilized to provide other visual cues on the wearer-facing surface and/or the garment-facing surface. As an example, the topsheet, or a layer subjacent to the topsheet may comprise colorant. For example, the topsheet may comprise colorant on an underside thereof. In combination or independently thereof, a subjacent layer to the topsheet may comprise colorant.

In one specific example, the backsheet may comprise colorant. This colorant may be disposed on a wearer-facing side of the backsheet and/or a garment-facing surface of the backsheet. In order to provide the placement cue, colorant on the backsheet can be provided in a first portion and a second portion. The first portion of colorant may be disposed on a first side of the absorbent core zone while the second portion of colorant may be disposed on a second side of the absorbent core zone opposite from the first side. A space which exists between the first portion and the second portion (the absorbent core zone) may be devoid of colorant, may have a lower basis weight of colorant than the first portion and/or second portion, and/or may have a contrasting color from those of the first portion and/or second portion.

The space between the first portion and the second portion extends generally in the longitudinal direction and may extend the full length of the absorbent article or may be shorter than the full length of the absorbent article. As an example, the space may have a length that is at least 50 percent of the overall length of the absorbent article, more preferably at least 75 percent, or most preferably at least 85 percent. In one specific example, the space between the first portion and the second portion may extend the full length of the absorbent article. In order to accommodate registration of the absorbent core and the absorbent core zone, inner edges of the first portion of colorant and/or the second portion of colorant may comprise a scalloped, sinusoidal or any other shape with curved designs of repeating elements. During manufacturing, the placement of the absorbent core, and therefore the absorbent core zone, in relation to the first portion and the second portion, may shift from product to product due to web tracking in a cross-machine direction (transverse direction). The shape of the inner edges of the first portion and second portion can accommodate this shifting of the absorbent core zone. Of import however, is the amplitude (height) of the crests of the curved design as well as the frequency (crests per cm) of the curved design. If the height of the crests is too great, then the colorant disposed in the first portion and/or second portion can appear jagged and uncomfortable. Where the height of the crests is too low, the curved design may not accommodate the variability of the location of the absorbent core zone.

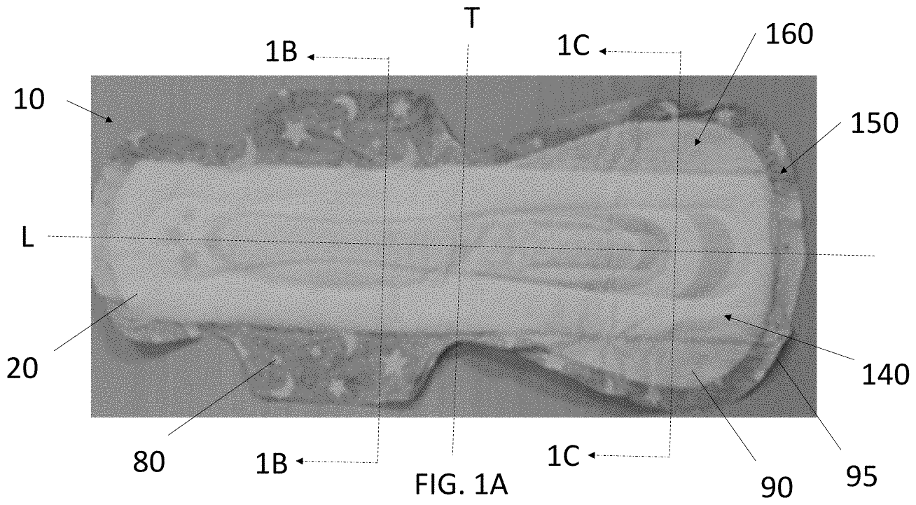

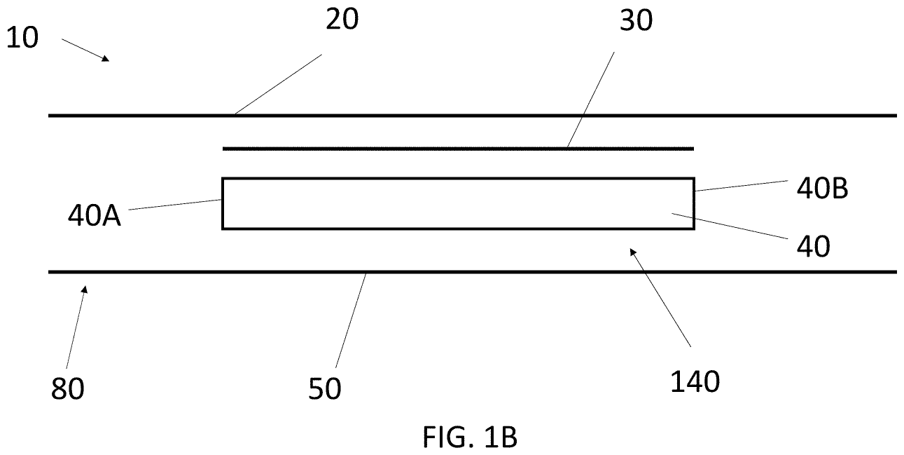

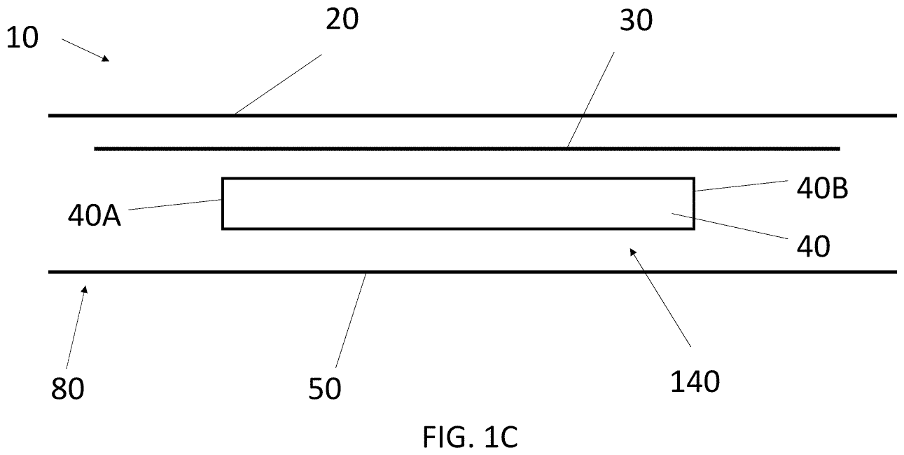

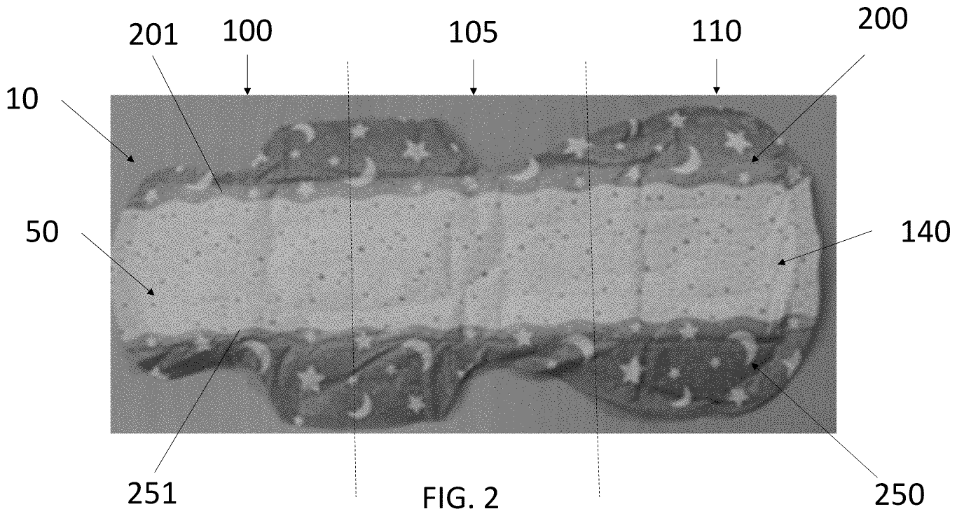

The above is further described in the following figures. As shown in FIGS. 1A-2, an absorbent article 10 comprises a longitudinal centerline “L,” a transverse centerline “T,” which is generally perpendicular to the longitudinal centerline L. And as noted previously, the absorbent article 10 comprises a front portion 100, a rear portion 110, and a central portion 105 disposed between the front portion 100 and the rear portion 110.

The absorbent article 10 further comprises a topsheet 20, an optional layer 30, an absorbent core 40, and a backsheet 50. Recall that the optional layer 30 may comprise an acquisition/distribution layer, separate acquisition and primary distribution layers, and/or a support layer. Additionally optional layer 30, although depicted between the topsheet 20 and the absorbent core 40, may be disposed between the backsheet 50 and the absorbent core 40. In such forms, the optional layer may comprise a secondary distribution layer and/or a support layer. Forms are also contemplated where optional layers are provided between the topsheet 20 and the absorbent core 40 as well as between the absorbent core 40 and the backsheet 50.

Additionally, the absorbent article 10 may comprise a primary pair of wings 80 on opposing sides of the absorbent article. As shown, the topsheet 20 and the backsheet 50 may extend transversely outward and form at least a portion of the primary wings 80. However, the primary wings 80 may be formed from discrete material as well. As an example, a discrete nonwoven material may be joined to the topsheet 20 and/or the backsheet 50 or may be attached somewhere between the topsheet 20 and the backsheet 50 and extend outward therefrom to form the primary wings 80. Forms are also contemplated where the discrete material along with the topsheet 20 and/or backsheet 50 are joined together to form the primary pair of wings 80.

As shown specifically in FIG. 1B, the optional layer 30 may be transversely co-extensive with the absorbent core 40. This particularly may be the case in the front portion 100 and at least part of the central portion 105. However, as shown in FIG. 1C, the optional layer 30 may extend transversely outboard of longitudinal side edges 40A and 40B of the absorbent core 40, particularly in the rear portion 110. The portion of the optional layer 30 which extends transversely beyond the longitudinal side edges 40A and 40B, is disposed in the inner boundary zone 160. As a reminder, the optional layer 30 is one of several options which can be utilized. The other options provided may have a construction similar to that shown in FIG. 1C regarding the transverse extent of the optional layer 30 and may be configured as described herein.

Based on the configuration shown in FIGS. 1B and 1C, an inner boundary zone 160 may be disposed primarily in the rear portion 110 and slightly in the central portion 105. And as shown, the front region 100 may not comprise an inner boundary zone 160. This type of configuration may be beneficial particularly in absorbent articles which are meant for overnight wear. As an example, overnight pads typically have a much longer length than their daily use counterparts. During the evenings, particularly when used during slumber, liquid insults to the pad tend to flow toward the rear portion of the article. So, the longer length of the overnight pads is believed to provide additional capacity to address these liquid insults. This makes the capacity cue (highlighting the edges of the absorbent core) in the rear portion extremely useful.

Referring again to FIGS. 1A-2, an outer periphery 95 is disposed where the topsheet 20 and the backsheet 50 are joined to one another. An outer boundary zone 150 comprises the outer periphery 95 and extends inboard toward the absorbent article. An absorbent core zone 140 is defined by the absorbent core 40 and is bounded by the longitudinal side edges 40A and 40B as well as end edges of the absorbent core 40.

Disposed between the outer boundary zone 150 and the absorbent core zone 140 is the inner boundary zone 160. As shown, the inner boundary zone 160 may form a portion of a secondary pair of wings 90 which extend transversely outward from the absorbent core zone 140. It is worth noting that the secondary pair of wings 90 is typical of overnight pads. So, for absorbent articles which are not overnight pads, the secondary pair of wings 90 is optional.

Referring now to FIG. 2, in order to accommodate the cues mentioned herein, the backsheet 50 may comprise colorant. As shown, the backsheet 50 may comprise a first colorant portion 200 and a second colorant portion 250. The first colorant portion 200 and the second colorant portion 250 may be disposed on opposite sides of the absorbent core zone 140.

Each of the first colorant portion 200 and second colorant portion 250 extend transversely outward from the absorbent core zone 140. Additionally, each of the first colorant portion 200 and the second colorant portion 250 comprise an inner edge 201 and 251, respectively. As discussed previously, the inner edges comprise a wave shape. The wave shape of the inner edges 201 and 251 can accommodate the variability of the location of the absorbent core zone 140.

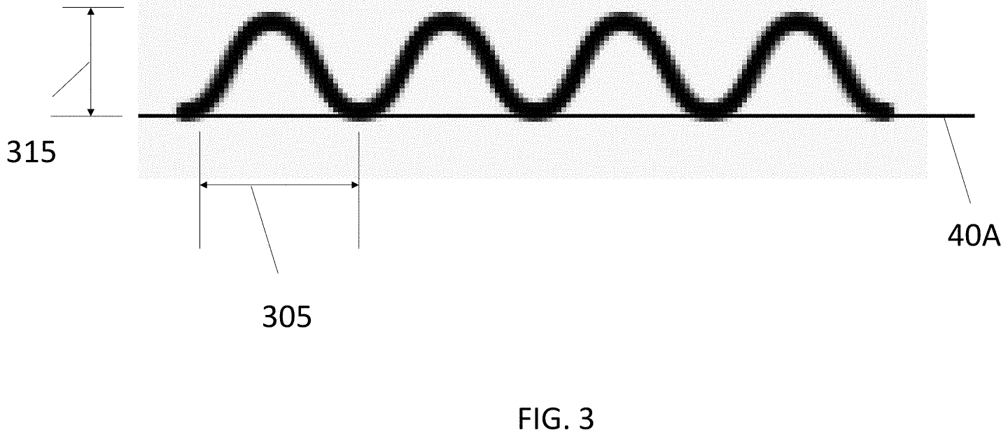

Regarding the curved design of the first portion and/or second portion, we refer to FIG. 3. In one specific example, the curved design may comprise a width 305 of a base that is 2 times or more than a height 315 of a crest. Additionally, an edge of the curved design may cover the variation of the absorbent core placement, when the curved design is off set from the longitudinal side edges of the absorbent core, e.g. 40A, by an amount equal the total placement variation minus 25 percent of the width of the base.

For the specific components of the absorbent article, any suitable materials may be utilized so long as they are in accordance with the requirements disclosed herein. For example, any suitable topsheet may be utilized so long as the opacity of the selected material meets the requirements described herein. Additionally, the topsheet may be compliant, soft feeling, and non-irritating to the wearer's skin. Suitable topsheet materials include a liquid pervious material that is oriented towards and contacts the body of the wearer permitting bodily discharges to rapidly penetrate through it without allowing fluid to flow back through the topsheet to the skin of the wearer. The topsheet, while being capable of allowing rapid transfer of fluid through it, may also provide for the transfer or migration of the lotion composition onto an external or internal portion of a wearer's skin.

A suitable topsheet can be made of various materials such as woven and nonwoven materials; apertured film materials including apertured formed thermoplastic films, apertured plastic films, and fiber-entangled apertured films; hydro-formed thermoplastic films; porous foams; reticulated foams; reticulated thermoplastic films; thermoplastic scrims; or combinations thereof.

Apertured film materials suitable for use as the topsheet include those apertured plastic films that are non-absorbent and pervious to body exudates and provide for minimal or no flow back of fluids through the topsheet. Nonlimiting examples of other suitable formed films, including apertured and non-apertured formed films, are more fully described in U.S. Pat. No. 3,929,135, issued to Thompson on Dec. 30, 1975; U.S. Pat. No. 4,324,246, issued to Mullane et al. on Apr. 13, 1982; U.S. Pat. No. 4,342,314, issued to Radel et al. on Aug. 3, 1982; U.S. Pat. No. 4,463,045, issued to Ahr et al. on Jul. 31, 1984; U.S. Pat. No. 5,006,394, issued to Baird on Apr. 9, 1991; U.S. Pat. No. 4,609,518, issued to Curro et al. on Sep. 2, 1986; and U.S. Pat. No. 4,629,643, issued to Curro et al. on Dec. 16, 1986.

Nonlimiting examples of woven and nonwoven materials suitable for use as the topsheet include fibrous materials made from natural fibers, e.g. cotton, including 100 percent organic cotton, modified natural fibers, synthetic fibers, or combinations thereof. These fibrous materials can be either hydrophilic or hydrophobic, but it is preferable that the topsheet be hydrophobic or rendered hydrophobic. As an option, portions of the topsheet can be rendered hydrophilic, using any known method for making topsheets containing hydrophilic components. Nonwoven fibrous topsheets 20 may be produced by any known procedure for making nonwoven webs, nonlimiting examples of which include spunbonding, carding, wet-laid, air-laid, meltblown, needle-punching, mechanical entangling, thermo-mechanical entangling, and hydroentangling.

The topsheet may be formed from a combination of an apertured film and a nonwoven. For example, a film web and a nonwoven web can be combined as described in U.S. Pat. No. 9,700,463. Alternatively, a film may be extruded onto a nonwoven material which is believed to provide enhanced contact between the film layer and the nonwoven material. Exemplary processes for such a combination are described in U.S. Pat. Nos. 9,849,602 and 9,700,463.

Additionally, the basis weight of these materials can be less than 40 gsm, more preferably less than 35 gsm, or most preferably less than 30 gsm. As another example, the basis weight of the topsheet material can be between about 15 gsm to about 40 gsm, more preferably from about 15 gsm to about 35 gsm, or most preferably from about 15 gsm to about 30 gsm, specifically including any values within these ranges and any ranges created thereby.

The backsheet may be positioned adjacent a garment-facing surface of the absorbent core and may be joined thereto by attachment methods such as those well known in the art. For example, the backsheet may be secured to the absorbent core and/or any layers therebetween by a uniform continuous layer of adhesive, a patterned layer of adhesive, or an array of separate lines, spirals, or spots of adhesive. Alternatively, the attachment methods may comprise using heat bonds, pressure bonds, ultrasonic bonds, dynamic mechanical bonds, or any other suitable attachment methods or combinations of these attachment methods as are known in the art.

The backsheet may be impervious, or substantially impervious, to liquids (e.g., urine) and may be manufactured from a thin plastic film, although other flexible liquid impervious materials may also be used. As used herein, the term “flexible” refers to materials which are compliant and will readily conform to the general shape and contours of the human body. The backsheet may prevent, or at least inhibit, the exudates absorbed and contained in the absorbent core from wetting articles of clothing which contact the incontinence pad such as undergarments. However, the backsheet may permit vapors to escape from the absorbent core (i.e., is breathable) while in some cases the backsheet may not permit vapors to escape (i.e., non-breathable). Thus, the backsheet may comprise a polymeric film such as thermoplastic films of polyethylene or polypropylene. A suitable material for the backsheet is a thermoplastic film having a thickness of from about 0.012 mm (0.5 mil) to about 0.051 mm (2.0 mils), for example. Any suitable backsheet known in the art may be utilized with the present invention.

The backsheet acts as a barrier to any absorbed bodily fluids that may pass through the absorbent core to the garment surface thereof with a resulting reduction in risk of staining undergarments or other clothing. A preferred material is a soft, smooth, compliant, liquid and vapor pervious material that provides for softness and conformability for comfort, and is low noise producing so that movement does not cause unwanted sound.

Exemplary backsheets are described in U.S. Pat. No. 5,885,265 (Osborn, III.) issued Mar. 23, 1999; U.S. Pat. No. 6,462,251 (Cimini) issued Oct. 8, 2002; U.S. Pat. No. 6,623,464 (Bewick-Sonntag) issued Sep. 23, 2003 or U.S. Pat. No. 6,664,439 (Arndt) issued Dec. 16, 2003. Suitable dual or multi-layer breathable backsheets for use herein include those exemplified in U.S. Pat. Nos. 3,881,489, 4,341,216, 4,713,068, 4,818,600, EP 203 821, EP 710 471, EP 710 472, and EP 793 952.

Suitable breathable backsheets for use herein include all breathable backsheets known in the art. In principle there are two types of breathable backsheets, single layer breathable backsheets which are breathable and impervious to liquids and backsheets having at least two layers, which in combination provide both breathability and liquid imperviousness. Suitable single layer breathable backsheets for use herein include those described for example in GB A 2184 389, GB A 2184 390, GB A 2184 391, U.S. Pat. Nos. 4,591,523, 3,989,867, 3,156,242 and WO 97/24097.

The backsheet may be a nonwoven web having a basis weight between about 20 gsm and about 50 gsm. As an example, the backsheet can be a relatively hydrophobic 23 gsm spunbonded nonwoven web of 4 denier polypropylene fibers available from Fiberweb Neuberger, under the designation F102301001. The backsheet may be coated with a non-soluble, liquid swellable material as described in U.S. Pat. No. 6,436,508 (Ciammaichella) issued Aug. 20, 2002.

The backsheet has a garment-facing side and an opposite body-facing side. The garment-facing side of the backsheet comprises a non-adhesive area and an adhesive area. The adhesive area may be provided by any conventional means. Pressure sensitive adhesives have been commonly found to work well for this purpose.

The topsheet may be joined to the backsheet by attachment methods (not shown) such as those well known in the art. The topsheet and the backsheet may be joined directly to each other in the article periphery and may be indirectly joined together by directly joining them to the absorbent core, the fluid management layer, and/or additional layers disposed between the topsheet and the backsheet. This indirect or direct joining may be accomplished by attachment methods which are well known in the art.

The absorbent core may comprise any suitable shape including but not limited to an oval, a discorectangle, a rectangle, an asymmetric shape, and an hourglass. For example, in some forms of the present invention, the absorbent core may comprise a contoured shape, e.g. narrower in the intermediate region than in the end regions. As yet another example, the absorbent core may comprise a tapered shape having a wider portion in one end region of the pad which tapers to a narrower end region in the other end region of the pad. The absorbent core may comprise varying stiffness in the MD and CD.

The configuration and construction of the absorbent core may vary (e.g., the absorbent core may have varying caliper zones, a hydrophilic gradient, a superabsorbent gradient, or lower average density and lower average basis weight acquisition zones). Further, the size and absorbent capacity of the absorbent core may also be varied to accommodate a variety of wearers. However, the total absorbent capacity of the absorbent core should be compatible with the design loading and the intended use of the disposable absorbent article or incontinence pad.

In some forms of the present disclosure, the absorbent core may comprise a plurality of multi-functional layers that are in addition to the first and second laminates. For example, the absorbent core may comprise a core wrap (not shown) useful for enveloping the first and second laminates and other optional layers. The core wrap may be formed by two nonwoven materials, substrates, laminates, films, or other materials. In a form, the core wrap may only comprise a single material, substrate, laminate, or other material wrapped at least partially around itself. The absorbent core may comprise one or more adhesives, for example, to help immobilize the SAP or other absorbent materials within the first and second laminates.

Absorbent cores comprising relatively high amounts of SAP with various core designs are disclosed in U.S. Pat. No. 5,599,335 to Goldman et al., EP 1,447,066 to Busam et al., WO 95/11652 to Tanzer et al., U.S. Pat. Publ. No. 2008/0312622A1 to Hundorf et al., and WO 2012/052172 to Van Malderen. These may be used to configure the superabsorbent layers.

Additions to the core of the present disclosure are envisioned. In particular, potential additions to the current multi-laminate absorbent core are described in U.S. Pat. No. 4,610,678, entitled “High-Density Absorbent Structures” issued to Weisman et al., on Sep. 9, 1986; U.S. Pat. No. 4,673,402, entitled “Absorbent Articles With Dual-Layered Cores”, issued to Weisman et al., on Jun. 16, 1987; U.S. Pat. No. 4,888,231, entitled “Absorbent Core Having A Dusting Layer”, issued to Angstadt on Dec. 19, 1989; and U.S. Pat. No. 4,834,735, entitled “High Density Absorbent Members Having Lower Density and Lower Basis Weight Acquisition Zones”, issued to Alemany et al., on May 30, 1989. The absorbent core may further comprise additional layers that mimic the dual core system containing an acquisition/distribution core of chemically stiffened fibers positioned over an absorbent storage core as detailed in U.S. Pat. No. 5,234,423, entitled “Absorbent Article With Elastic Waist Feature and Enhanced Absorbency” issued to Alemany et al., on Aug. 10, 1993; and in U.S. Pat. No. 5,147,345. These are useful to the extent they do not negate or conflict with the effects of the below described laminates of the absorbent core of the present invention. Additional examples of suitable absorbent cores are described in U.S. Patent Application Publication Nos. 2018/0098893 and 2018/0098891.

Regarding the optional layers of absorbent system, any suitable material may be utilized. Each of these layers may have a basis weight from about 40 gsm to about 100 gsm, from about 45 gsm to about 75 gsm, or from about 50 gsm to about 65 gsm, specifically including all values within these ranges and any ranges created thereby. In some forms, the one or more of these layers may comprise a homogeneous mix of fibers whereas in other forms, the fluid management layer may comprise a heterogeneous mix of fibers.

Some exemplary optional layers for the absorbent system are described in U.S. Patent Application Publication Nos. 2015/0351976 A1 and 2014/0343523 A1; and U.S. patent application Ser. No. 15/729,704.

### Test Methods

### Color Measurement Method

Color analyses of various zones on an absorbent article are made using a 0°/45° spectrophotometer with adjustable apertures capable of making standard CIE L*a*b* measurements in accordance with ASTM E1349. An example of a suitable spectrophotometer is the Labscan XE (available from Hunter Associates Laboratory, Inc., Reston, Va., or equivalent). All testing is performed in a room maintained at a temperature of 23° C.±2.0° C. and a relative humidity of 50%±2% and test samples are conditioned under the same environmental conditions for at least 2 hours prior to testing.

For this procedure, the test sample is the intact absorbent article. A total of three individual test samples are required. Determine and note which edge of the sample will be oriented towards the front of the body when in use. Also note which side of the sample is intended to face the body in use. The wearer-facing side of the sample will face the aperture during the measurements. The sample is removed from any outer packaging, unfolded (if necessary), and any release paper that is present is peeled away and disposed. A light coating of talc is applied to the panty fastening adhesive and wing adhesive to mitigate tackiness. The sample is then divided into longitudinal thirds and marked to denote a front portion, a central portion and a rear portion, as follows. The sample is laid flat on a bench and the length of the sample is measured along the longitudinal axis at the transverse midpoint. To mark the front portion, a line that is parallel to the transverse axis is marked on the sample at a distance that is ⅓ the length of the sample away from the front edge. To mark and divide the central and rear portions, a line that is parallel to the transverse axis is marked on the sample at a distance that is ⅔ the length of the sample away from the front edge. The central portion is the section between the lines depicting ⅓ and ⅔ the length of the sample, and the rear portion is the section behind the line depicting ⅔ the length of the sample. In like fashion, draw lines to denote the front, central and rear portions on each of the three samples.

Prior to making any color measurements, the spectrophotometer is calibrated and standardized per the vendor instructions using the standard white and black tiles provided by the vendor. The spectrophotometer must be standardized for each different size aperture just prior to making measurements with each respective aperture. Set the spectrophotometer to use the CIE L*a*b* color space with a D65 standard illumination, a 10° observer, and the UV filter set to nominal. The appropriate size aperture is selected according to which sample zone is being measured, further described herein. The test location for each sample zone is placed flat and centered over the aperture such that the entire aperture is covered by the test location. The standard white tile is held in place on the back side of the test location and a reading is taken. The L*, a*, and b* values are recorded to the nearest 0.01 units.

Referring to FIG. 1A, there are three different zones on the test sample that will undergo color measurements. Zone 1 is the Absorbent Core Zone 140, Zone 2 is the Outer Boundary Zone 150 and Zone 3 is the Inner Boundary Zone 160.

For Zone 1 (absorbent core zone 140), color measurements can be obtained from the front, central or rear portions of the sample, however the test location must be free from added color (e.g print signal or other region of color) with wrinkles and folds avoided. For Zone 1, the aperture size is 0.7 inch in diameter with a 0.50 inch diameter area view. Color readings are obtained from Zone 1 on each of the three test samples and recorded to the nearest 0.01 units. Calculate the arithmetic mean for the L*, a* and b* values obtained across all replicates and report as L*₁, a*₁ and b*₁ to the nearest 0.01 units.

For Zone 3 (inner boundary zone 160), color is measured at a test location that is also free of wrinkles or folds. Zone 3 will most likely reside in the rear portion of the sample. For Zone 3, the aperture size is 0.70 inch in diameter with a 0.50 inch diameter area view. Color readings are obtained from Zone 3 on each of the three test samples and recorded to the nearest 0.01 units. Calculate the arithmetic mean for the L*, a* and b* values obtained across all replicates and report as L*₃, a*₃ and b*₃ to the nearest 0.01 units.

For Zone 2 (outer boundary zone 150), color measurements are recorded to the nearest 0.01 units at multiple test locations in the rear portion of the sample. Due to the small size area in Zone 2, the aperture size is 0.40 inch in diameter with a 0.25 inch diameter area view. The first color reading for Zone 2 is obtained at the first 10 mm location just behind the line that depicts ⅔ the sample length in the rear portion of the sample starting at one of the lateral sides. Subsequent color readings are then taken every 10 mm as the aperture is walked along the entire rear portion of the sample in Zone 2 until the line that depicts ⅔ the sample length is reached on the opposite lateral side of the sample. In like fashion, color readings are obtained from Zone 2 on each of the three test samples and recorded to the nearest 0.01 units. Calculate the arithmetic mean for the L*, a* and b* values obtained across all replicates and report as L*₂, a*₂ and b*₂ to the nearest 0.01 units.

The color difference between the color measurements obtained for the different Zones is calculated as Delta E*. To determine the color difference between Zone 1 (absorbent core zone 140) and Zone 3 (inner boundary zone 160), calculate Delta E* as follows and report to the nearest 0.01 unit, noting the two zones that are being compared:

Delta E*=Sqrt[(L₃*−L₁*)²+(a₃*−a₁*)²+(b₃*−b₁*)²]

To determine the color difference between Zone 2 (outer boundary 150) and Zone 3 (inner boundary 160), calculate Delta E* as follows and report to the nearest 0.01 unit, noting the two zones that are being compared:

Delta E*=Sqrt[(L₃*−L₂*)²+(a₃*a₂*)²+(b₃*−b₂*)²]

### Opacity Measurement Method

Opacity measurements are made using a 0°/45° spectrophotometer with adjustable apertures capable of making standard CIE color measurements using XYZ coordinates and contrast ratio. An example of a suitable spectrophotometer is the Labscan XE (available from Hunter Associates Laboratory, Inc., Reston, Va., or equivalent). Measurements are conducted on either a single layer or multiple layers of test materials. All testing is performed in a room maintained at a temperature of 23° C.±2.0° C. and a relative humidity of 50%±2% and samples are conditioned under the same environmental conditions for at least 2 hours prior to testing.

Obtain a test sample by removing it from an absorbent article, if necessary. When excising the sample from an absorbent article, use care to not impart any contamination or distortion to the sample layer during the process. The test sample is obtained from an area free of folds or wrinkles, and it must be larger than the aperture being used on the spectrophotometer. Note which side of the test sample is facing (or is meant to face) the body during use. If more than one type of material is to be measured as multiple layers, note the side of each layer that is facing (or is meant to face) the body during use. The wearer-facing surface of the sample(s) is the side that will face the aperture during the measurement. Obtain a sufficient quantity of the sample material(s) such that ten measurements can be made on non-overlapping areas of the material(s) being evaluated.

To measure Opacity, calibrate and standardize the instrument per the vendor instructions using the standard white and black tiles provided by the vendor with a 0.70 inch diameter aperture in place. Set the spectrophotometer to use the CIE XYZ color space with a D65 standard illumination, a 10° observer, 0.70 inch aperture, 0.50 inch area view, and set the UV filter to nominal. Place the body-facing side of the test sample over the aperture and ensure that the entire aperture opening is covered by the sample. If measuring multiple layers, stack them on top of the body-facing layer according to their placement in the absorbent article, centered over the aperture, with the body-facing side of each oriented towards the aperture. Place the standard white tile directly against the back side of the sample(s), take a reading and record the Y value as Y_{white backing} to the nearest 0.01 units. Without moving the position of the test sample(s), remove the standard white tile and replace it with the black standard tile. Take a reading and record the Y value as Y_{black backing} to the nearest 0.01 units. Calculate Opacity by dividing the Y_{black backing} value by the Y_{white backing} value and then multiply by 100. Record Opacity to the nearest 0.1 percent, noting the material (or materials) that were measured.

In like fashion, repeat for a total of five measurements on non-overlapping areas of the test sample material(s). Calculate the arithmetic mean for Opacity obtained across all and report to the nearest 0.1 percent, noting the material (or materials) that were measured.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A disposable absorbent article having a longitudinal axis and a transverse axis disposed perpendicularly to the longitudinal axis, a front portion, a rear portion, and a central portion disposed between the front portion and rear portion, the disposable absorbent article further comprising:
a topsheet;
a backsheet;
an absorbent system disposed between the topsheet and the backsheet, wherein the absorbent system comprises an absorbent core having longitudinal side edges on opposite sides of the absorbent core and end edges on opposite sides of the absorbent core, the absorbent core being disposed in an absorbent core zone, wherein the absorbent core zone exhibits a first L* value;
an outer periphery formed at least in part by a portion of the topsheet and a portion of the backsheet;
an outer boundary zone comprising the outer periphery, the outer boundary zone having a width of less than 30 mm, wherein the outer boundary zone exhibits a second L* value of less than 89;
an inner boundary zone disposed between the outer boundary zone and the absorbent core zone in the rear portion, the inner boundary zone exhibits a third L* value which is greater than the second L* value; and
wherein the first L* value is greater than the second and third L* values such that there is a perceptible visual difference between the absorbent core zone and the inner boundary zone.

2. The absorbent article of claim 1, wherein the second L* value is from between about 50 and 89.

3. The absorbent article of claim 1, wherein the third L* value is from between about 60 and 94.

4. The absorbent article of claim 1, wherein a first delta E* value between the absorbent core zone and the inner boundary zone is greater than 4, more preferably greater than 7, or most preferably greater than 9.

5. The absorbent article of claim 1, wherein a second delta E* value between the inner boundary zone and the outer boundary zone is greater than 4, more preferably greater than 6, or most preferably greater than 8.

6. The absorbent article of claim 5, wherein the first delta E* is less than the second delta E* value.

7. The absorbent article of claim 1, wherein colorant is disposed on a surface of the topsheet.

8. The absorbent article of claim 1, wherein colorant is disposed on a surface of the backsheet.

9. The absorbent article of claim 1, wherein the topsheet has an opacity of less than 53, more preferably less than 51, or most preferably less than 47.

10. The absorbent article of claim 1, wherein the topsheet comprises a nonwoven material.

11. The absorbent article of any of the preceding claims, further comprising a primary pair of wings extending transversely outward from the absorbent core zone.

12. The absorbent article of claim 1, further comprising a secondary pair of wings extending transversely outward from the absorbent core zone, and wherein the secondary pair of wings are disposed in the rear portion of the absorbent article.

13. The absorbent article of claim 12, wherein the inner boundary zone forms a portion of the secondary pair of wings.

14. The absorbent article of claim 1, wherein the backsheet comprises a colorant, and wherein the colorant is disposed on opposing sides of the absorbent core zone.

15. The absorbent article of claim 14, wherein a first colorant portion is disposed on a first side of the absorbent core zone, and a second colorant portion is disposed on a second side of the absorbent core zone.

16. The absorbent article of claim 15, wherein a longitudinal edge of the first colorant portion adjacent the absorbent core zone has a wave shape of repeating crests and troughs.

17. The absorbent article of claim 1, further comprising an optional layer disposed between the topsheet and the absorbent core or between the absorbent core and the backsheet.

18. The absorbent article of claim 17, wherein the optional layer is disposed in the absorbent core zone and in the inner boundary zone, but not the outer boundary zone.

19. The absorbent article of claim 17, wherein the optional layer comprises an acquisition/distribution layer or an acquisition layer and a separation distribution layer.

20. The absorbent article of claim 19, wherein the acquisition/distribution layer or acquisition layer and distribution layer are disposed between the topsheet and the absorbent core.
